(19) United States
(12) Patent Application Publication
(10) Pub. No.: US 2004/0154623 A1
(43) Pub. Date: Aug. 12, 2004

(54) Tracheostomy tube and loading dilator

(75) Inventors: Darin G. Schaeffer, Bloomington, IN (US); Daniel J. Sirota, Bloomington, IN (US)

Correspondence Address: BRINKS HOFER GILSON & LIONE, ONE INDIANA SQUARE, SUITE 1600, INDIANAPOLIS, IN, 46204, US

(73) Assignee: Cook Critical Care

(21) Appl. No.: 10/769,962

(22) Filed: Feb. 2, 2004

Related U.S. Application Data
(60) Provisional application No. 60/444,578, filed on Feb. 3, 2003.
(60) Provisional application No. 60/517,593, filed on Nov. 5, 2003.

(51) Int. Cl. A61M 16/00

(52) U.S. Cl. 128/207.14

(57) ABSTRACT

A tracheostomy tube having a flange selectively attachable to and detachable from the tubular body. Utilizing an attachable flange that is not attached to the tracheostomy tube body until the flange has been inserted into the trachea enables the tracheostomy tube to be inserted by way of a radially expandable introducer sheath, thereby preventing substantial deformation of the body being dilated in the direction of movement of a dilator. In another embodiment, a tracheostomy tube/loading dilator combination is provided. The loading dilator has a larger-diameter proximal portion and a smaller diameter distal portion tapered at its distal end. The smaller-diameter distal portion is sized for insertion through the longitudinal bore of the tracheostomy tube such that the tapered distal portion extends axially beyond the tapered distal tip of the tracheostomy tube. The tracheostomy tube further includes a stop portion at its proximal end for engaging a distal portion of the larger-diameter proximal portion of the dilator, to limit axial movement of the loading dilator through the tracheostomy tube. Additionally, a tracheostomy tube/dilator combination may be provided having a locking assembly. In a further embodiment, a trimmable tracheostomy tube having a variable length is provided, such that the tracheostomy tube may be adjusted for use with patients of various size.

### RELATED APPLICATIONS

The present patent document claims the benefit of the filing dates under 35 U.S.C. § 119(e) of Provisional U.S. Patent Application Serial No. 60/444,578, filed Feb. 3, 2003, and 60/517,593, filed Nov. 5, 2003, the disclosures of which are hereby incorporated by reference.

### BACKGROUND

This application relates to a tracheostomy tube, and to a tracheostomy tube/loading dilator system.

In certain medical procedures, such as a tracheostomy procedure, physicians and other medical professionals may encounter difficulties when attempting to insert medical instruments, such as a tracheostomy tube, through a pre-dilated hole in a patient's body. During a percutaneous tracheostomy, a dilator is advanced into the trachea through a pre-dilated hole in order to open the trachea for insertion of a tracheostomy tube. A problem with this procedure is that the axial force exerted by the dilator on the trachea may result in the collapse of the trachea, thereby significantly increasing the trauma to the patient. In order to avoid tracheal collapse, the physician must slowly and repeatedly insert a dilator, or a series of dilators of increasing diameter, to incrementally greater depths in the trachea until the desired dilated diameter is obtained.

Some dilators, such as the Cook® Ciaglia Blue Rhino™ dilator, have hydrophilic coatings which are effective for reducing the friction encountered while dilating the trachea in this manner, and thereby reducing the amount of axial force exerted on the trachea. Although the use of such coated dilators is generally effective for reducing the amount of friction encountered during dilation, there remains a desire to even further reduce the forces exerted on the trachea during dilation, thereby further minimizing the risk of trauma to the patient.

One manner of addressing the problem of tracheal collapse is discussed in U.S. patent application Ser. No. 10/608,043, titled INTRODUCER SHEATH, filed Jun. 27, 2003, and incorporated by reference herein. In this application an introducer sheath is employed to provide a barrier between a patient's body and the dilator during insertion of the dilator into the body. The introducer sheath, supported externally from the body, is expandable in the radial direction upon insertion of a dilator. This radial expansion offsets and thereby reduces the axially-directed force applied by the dilator.

When the introducer sheath of the incorporated-by-reference patent application is used for the introduction of a medical device, such as a tracheostomy tube, that has a flange or other extension member projecting radially from the body of the device, difficulties can be encountered when attempting to withdraw the introducer sheath from the body opening following insertion of the medical device. In the case of a tracheostomy tube, the flange portion of the tube is situated directly in the path of the withdrawing sheath, thereby obstructing the withdrawal of the sheath. It is desired to provide a medical device in which the radially-extended portion may be selectively removed or otherwise manipulated in a manner that facilitates withdrawal of the sheath.

Another problem that may be encountered in medical procedures is that a dilator or other medical device may be inadvertently inserted too far into the body. With particular reference to a tracheostomy procedure, this problem may occur if the distal end of the dilator is inadvertently extended too far into the trachea such that it punctures the trachea posterior wall, i.e., that portion of the trachea wall directly across from the entry site into the trachea. It is desired to provide a mechanism that allows the dilator to protrude a fixed amount, and no more, from the distal end of the tracheostomy tube so that the operator can effectively monitor the penetration of the dilator and thereby prevent excess penetration of the device into the trachea.

Difficulties may also be encountered in tracheostomy procedures when a tracheostomy tube having a removable or otherwise manipulatable flange is utilized. Since the flange of the tracheostomy tube is removed or otherwise manipulated out of its normal position at the time of insertion of the tube into the body, it is possible to inadvertently insert the tracheostomy tube too far into the trachea. When this occurs, there is no convenient way of retrieving the tube. It is desired to provide a mechanism to prevent inadvertent over-insertion of the tracheostomy tube.

Another problem that exists in tracheostomy procedures arises from the fact that, due to their very nature as critical care devices, such procedures must generally be performed as rapidly as possible with little advance notice. Due to the wide variance in size of patients, a tracheostomy tube of a size suitable for one patient may not suitable for another. Tracheostomy tubes of optimal size for each and every patient may not always be readily available, and therefore the physician must sometimes utilize the best available tracheostomy tube, even if it isn't optimal for the patient. It is desired to provide a tracheostomy tube that is variable in length to enable the tube to be used in a wider variety of patients than existing tubes.

### BRIEF SUMMARY

The foregoing problems are overcome and a technical advance is achieved in the inventive tracheostomy tube and a tracheostomy tube/loading dilator system.

In one embodiment, the present invention comprises a tracheostomy tube comprising a hollow tubular body having a proximal end portion, a distal end portion and a curved portion intermediate the proximal and distal end portions. A flange is situated at the proximal end portion. The flange is capable of radial extension from the tube, and is manipulatable to selectively prevent the radial extension. Preferably, the flange is selectively attachable to the proximal end portion to provide radial extension, and detachable from the proximal end portion when radial extension is to be prevented.

In another embodiment, the present invention comprises an insertion device. The insertion device comprises a tracheostomy tube and a loading dilator. The tracheostomy tube has a longitudinal bore and a tapered distal tip, and the loading dilator has a larger-diameter stepped proximal portion and a smaller diameter distal portion tapered at its distal end. The smaller-diameter distal portion is sized to be insertable through the longitudinal bore of the tracheostomy tube such that the tapered distal portion extends axially beyond the tapered distal tip of the tracheostomy tube. The tracheostomy tube has a proximal end and further comprises a stop portion at the proximal end for engaging a distal portion of the larger-diameter stepped portion of the dilator, to thereby limit axial movement of the loading dilator through the tracheostomy tube.

In yet another embodiment, the present invention comprises a device for percutaneous insertion into the trachea of a patient. The device comprises a tracheostomy tube having a longitudinal passageway therethrough. The tracheostomy tube has a distal end portion percutaneously insertable into the trachea and a proximal end portion exterior to the trachea when the distal end portion is inserted. The tracheostomy tube further has a radially extending flange attachable to the proximal end portion of the tracheostomy tube when the distal end portion is inserted into the trachea. A dilator is positionable within the longitudinal passageway of the tracheostomy tube for dilating an opening in the trachea for insertion of the tracheostomy tube. A locking assembly is provided for locking the tracheostomy tube to the dilator during insertion of the tracheostomy tube into the trachea.

In still another embodiment, the present invention comprises a method of inserting a tracheostomy tube into the trachea of a patient. A tracheostomy tube comprising a hollow tubular body having a longitudinal passageway therethrough is provided. The tubular body has a distal end portion for insertion into the trachea, and a proximal end portion exterior to the trachea when the distal end portion is inserted. After the distal end portion of the tubular body has been inserted into the trachea, an excess portion of the proximal end portion of said tubular body is trimmed away. The flange is then engaged with the tracheostomy tube at the proximal end portion of the tubular body.

### BRIEF DESCRIPTION OF THE DRAWINGS

FIG. 1 is a side view of a process for dilating a body opening illustrating the use of the radially-expandable sheath of U.S. patent application Ser. No. 10/608,043;

FIG. 2 is a side view of a prior art process for dilating a body opening;

FIG. 3 is a perspective view of a tracheostomy tube according to an embodiment of the present invention prior to attachment of a flange to the tube.

FIG. 4 is a perspective view of a flange that is attachable to the tracheostomy tube of FIG. 3.

FIG. 5 is a side view of a dilator according to an embodiment of the present invention.

FIG. 6 is a perspective view of a tracheal insertion device comprising the tracheostomy tube of FIG. 3 and the dilator of FIG. 5.

FIG. 7 is a perspective view of a dilator including apparatus for locking the dilator with a tracheostomy tube.

FIG. 8 is a perspective view of the dilator of FIG. 7 in combination with a tracheostomy tube, and illustrating a locking device for locking the tracheostomy tube to the dilator.

FIG. 9 is a sectional view of a portion of the tracheostomy tube/dilator combination of FIG. 8.

FIG. 10 is an embodiment of the present invention showing a tracheostomy tube having an inner cannula.

FIG. 11 shows a tracheostomy tube having a trimmable proximal portion.

FIG. 12 is a view of a collar and tracheostomy tube, partially in section, showing the presence of barbs on the inner surface of the collar.

### DETAILED DESCRIPTION OF THE DRAWINGS AND THE PRESENTLY PREFERRED EMBODIMENTS

The present invention in its various embodiments described hereinafter may be utilized in medical procedures, including but not limited to tracheostomy procedures. The present invention is particularly beneficial when utilized for tracheostomy procedures carried out using the radially expandable thin-wall introducer sheath described in the incorporated-by-reference application Ser. No. 10/608,043.

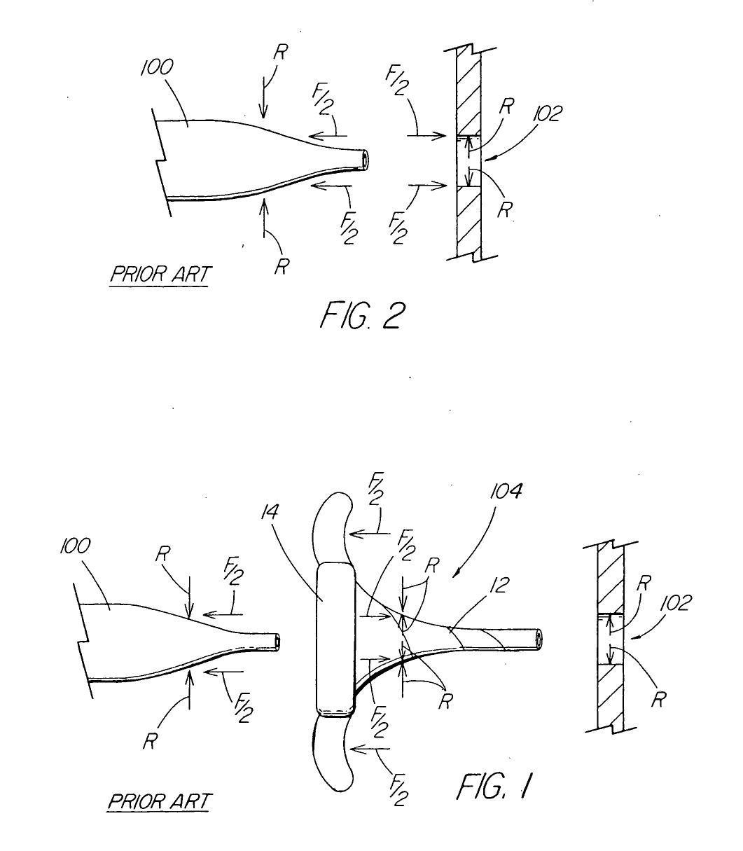

FIGS. 1 and 2 are provided to illustrate the use of the above-described radially expandable introducer sheath, and to provide background information to facilitate understanding of embodiments of the present invention. FIG. 1 shows a side view of a radially expandable thin-wall introducer sheath described in the incorporated-by-reference application. The introducer sheath 104 is positioned between a conventional dilator 100 and a body opening 102 of the patient that is being dilated for introduction of a medical device. As shown in the figure, the use of the introducer sheath allows the body being dilated to be primarily subjected to radial forces (R) transferred to the body, thereby preventing substantial deformation of the body in the direction of movement of the dilator. This occurs due to the radial expansion of the introducer sheath upon insertion of the dilator. Further details of this operation are provided in the incorporated-by-reference application.

For purposes of comparison, FIG. 2 illustrates the use of a conventional dilator 100 without the introducer sheath. In this instance, an axial force (F/2) is exerted on the body being dilated, causing axial deformation of the body in the direction of movement of the dilator. In addition to causing discomfort to the patient, the applied axial force may cause additional problems, such as the collapse of the trachea or body part being dilated.

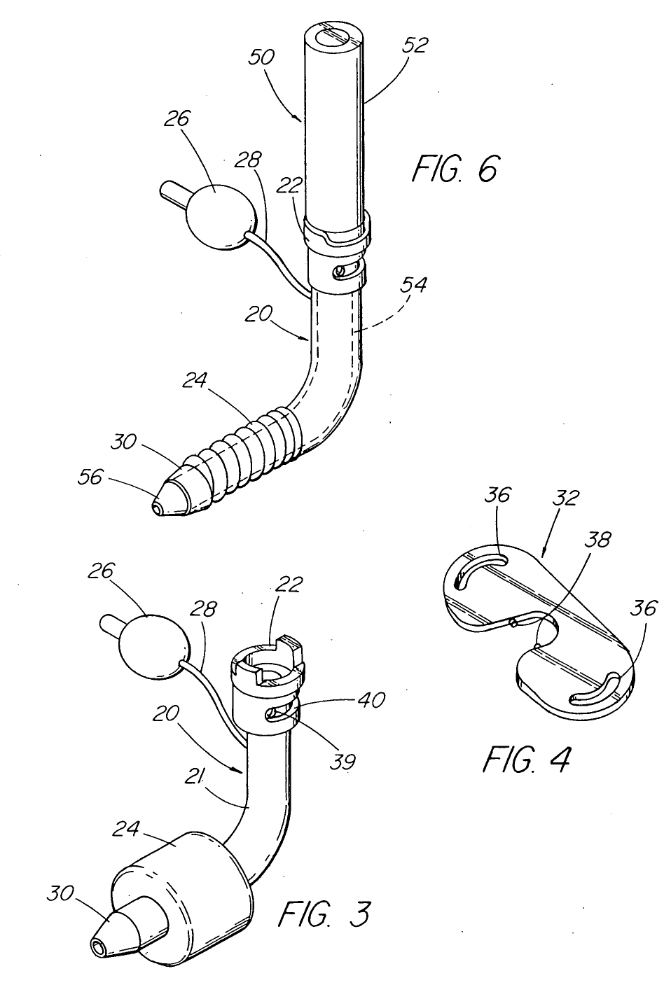

FIG. 3 is a perspective view of a tracheostomy tube 20 according to one embodiment of the present invention. In this embodiment, tracheostomy tube 20 includes a curved tubular body 21 and an optional collar 22 fitted over the proximal end of body 21. Tracheostomy tube 20 may also include a conventional inflatable tracheal cuff 24. Tracheostomy tube 20 also includes cuff inflation port 26, and an inflation line 28 running from inflation port 26 to cuff 24 to transmit an inflation fluid, such as air, from the port to the cuff in well-known fashion. Distal end 30 of tubular body 21 is tapered to provide a smooth transition with the distal tip of a dilator (not shown).

FIG. 4 is a perspective view of one embodiment of a flange 32 that is selectively attachable to, and removable from, tracheostomy tube 20. Tracheostomy tube flanges are well-known, and flange 32 may have any general shape commonly utilized for this purpose. In the embodiment shown, flange 32 includes a cut-away portion 34 and an opening 36 at each longitudinal end of the flange. According to the present invention, flange 32 includes apparatus for the selective attachment and removal of the flange from tracheostomy tube 20. In the embodiment shown, the attachment and removal apparatus comprises opposing snaps 38. When flange 32 is attached to tracheostomy tube 20, cut-away portion 34 mates with groove 40 on collar 22. Snaps 38 are snap-fit into complementary receptacles 39 in tracheostomy tube body 21 (only one of which is shown in the view of FIG. 3) to securely, but removably, affix flange 32 to tracheostomy tube 20. Once attached to the tracheostomy tube, the flange functions in the same manner as a non-removable flange on a conventional tracheostomy tube.

Although FIGS. 3 and 4 illustrate a snap-fit attachment mechanism, those skilled in the art will appreciate that other conventional attachment mechanisms may be readily substituted, and are considered within the scope of the invention. In addition, in some embodiments of the present invention the flange need not be fully removed from the tracheostomy tube. Rather, the flange can be axially folded against the body of the tracheostomy tube or otherwise manipulated in a manner such that it does not appreciably increase the diameter of the tracheostomy tube, so that the introducer sheath can be readily withdrawn in an axial direction over the tracheostomy tube.

Although the embodiment shown illustrates the use of collar 22 for facilitating and securing the snap-fit connection, the presence of a collar is not required for this connection. However, it is preferred to include a rigid or semi-rigid collar as shown, as it is believed that the presence of the collar improves and secures the attachment of the flange to the tracheostomy tube. When present, collar 22 can be formed integral with tracheostomy tube body 21 by well-known means, such as molding, or can be securely affixed to the proximal portion of the tracheostomy tube. As shown in FIG. 12, barbs 25 may be provided on the inner surface of collar 22 to provide a secure attachment of collar 22 with tube body 21. Barbs 25 may be provided on the inner surface of the collar by any conventional means, such as molding the barbs into the collar or snapping the barbs into a suitably-sized aperture in the collar. In a preferred embodiment, four sets of barbs (two of which are visible in the embodiment of FIG. 12) may be positioned roughly 90° apart along the inner circumference of collar 22. Alternatively, barbs may be placed on the outer surface of tube body 21 to provide said attachment with collar 22.

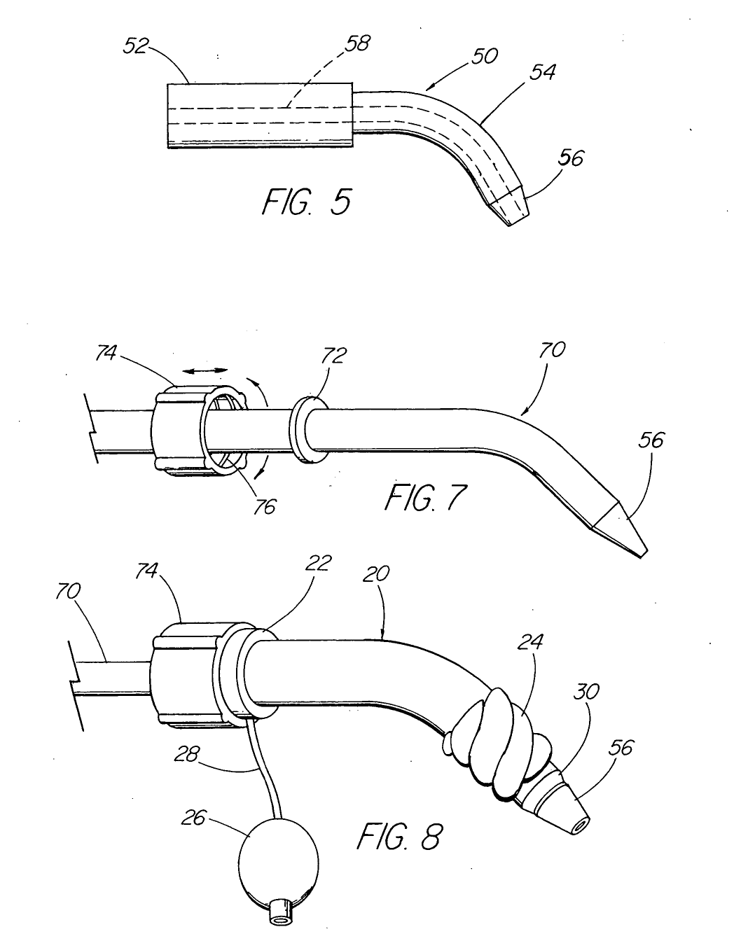

Another feature of the present invention is a stepped insertion (or loading) dilator 50 that may be used in connection with a tracheostomy tube to form an insertion device. One embodiment of a stepped dilator 50 according to the present invention is shown in FIG. 5. By “stepped” is meant that the dilator has a larger diameter, or “stepped”, proximal portion 52, and a smaller diameter main body portion 54 that extends in a distal direction from the large diameter portion. Stepped proximal portion 52 and main body portion 54 can be molded as integral components of insertion dilator 50. Alternatively, main body portion 54 may be a separate tubular component that is attached to proximal end portion 52, such as by sliding a proximal portion of main body portion 54 into a central longitudinal passageway of stepped portion 52 and bonding it thereto. In the embodiment shown, main body portion 54 is curved to match the contour of the tracheostomy tube into which it is to be inserted. Main body portion 54 terminates in a distal tip 56. The dilator includes a central lumen 58, shown in phantom in FIG. 5, that extends axially through the dilator in conventional fashion.

The presence of a larger diameter proximal portion 52 of dilator 50 also provides another advantage. Due to its large diameter, large diameter proximal portion 52 provides a convenient gripping surface. This allows the operator to firmly grasp the dilator so that the distal end of the dilator can be forcibly inserted through the body opening. Since the gripping/pushing surface is not inserted into the body, this surface can be formed from any convenient polymeric or other substance that is capable of forming a gripping surface. Non-limiting examples of suitable materials for forming the gripping surface include lower durometer urethane, thermoplastic rubber, or other well-known thermoplastic or non-thermoplastic elastomers.

FIG. 6 shows an embodiment wherein main body portion 54 of stepped insertion dilator 50 has been inserted into tracheostomy tube 20 in conventional fashion through the main lumen of the tracheostomy tube. The part of main body portion 54 of dilator 50 that is situated within tracheostomy tube 20 is shown in phantom. In this embodiment, the distal end of dilator proximal end portion 52 is sized to meet and abut collar 22 of tracheostomy tube 20 to obstruct further axial penetration of the dilator, thereby preventing the dilator from being advanced too far into the tracheostomy tube. If dilator distal tip 56 was not prevented from protruding an excessive distance from the distal end of the tracheostomy tube in this manner, the posterior wall of the patient's trachea could be inadvertently punctured.

The embodiment of FIG. 6 illustrates the presence of collar 22. However, as stated, the presence of a collar is not required in all embodiments. When a collar is not utilized, the dilator and the tracheostomy tube can be cooperatively sized and shaped in a manner such the proximal end of the tracheostomy tube has a diameter sufficient to obstruct and prevent further axial penetration of the dilator.

The tracheostomy tube can be conveniently inserted in proper position in the trachea using the combined tracheostomy tube/loading dilator of FIG. 6, and the radially expandable introducer sheath illustrated in FIG. 1. For insertion of the tracheostomy tube, the introducer sheath is initially introduced into a pre-dilated body opening. A dilator is then inserted as shown in FIG. 1 to provide a tracheal opening of designated size. The tracheostomy tube/loading dilator combination of FIG. 6 is then inserted through the tracheal opening. Once the tracheostomy tube is in proper position in the trachea, the introducer sheath is withdrawn.

When using a conventional tracheostomy tube having the flange attached thereto in conventional fashion, it would be difficult, if not impossible, to withdraw the introducer sheath from the body opening over the tracheostomy tube. The presence of the flange on a conventional tracheostomy tube acts to increase the effective diameter of the tracheostomy tube/dilator combination, thereby providing an obstruction to smooth withdrawal of the introducer. By utilizing a tracheostomy tube for insertion that has not yet had the flange attached, or from which the flange has been removed, axially folded or otherwise manipulated in a manner such that it no longer obstructs removal of the sheath, the introducer sheath can be readily withdrawn in an axial direction over the tracheostomy tube. Once the sheath has been withdrawn, the flange can be simply snapped, extended, or otherwise affixed to the tracheostomy tube.

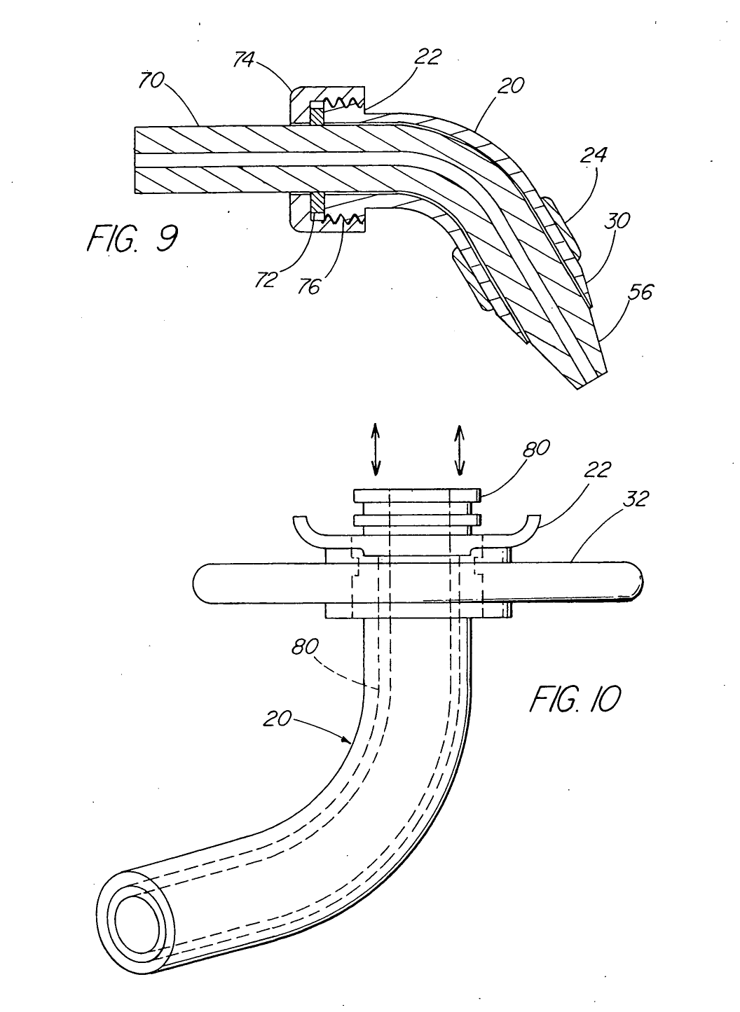

Another feature of the present invention is the combination of a loading dilator and a tracheostomy tube that have complementary tapered distal tips. Distal end 30 of the tracheostomy tube and distal tip 56 of the dilator are tapered such that they have a smooth diametrical transition when the loading dilator is inserted into the main lumen of the tracheostomy tube, as shown in FIG. 6. This smooth diametrical transition is also shown in FIG. 8, and in the cross-sectional view of FIG. 9. When the respective distal ends are tapered in this manner, the insertion device (tracheostomy tube/loading dilator), has a more tapered tip than found in conventional devices. This device can be used to dilate the trachea at the same time that the tracheostomy tube/loading dilator is being inserted into the trachea or other body opening, without requiring a pre-dilation step.

Another feature of the present invention comprises a locking mechanism for locking the dilator to the tracheostomy tube. When a tracheostomy tube as described herein that does not have a neck flange attached is inserted using an introducer sheath as described in the incorporated by reference application, it would be possible to inadvertently insert the tracheostomy tube too far into the body opening. Normally, the presence of the flange on the tracheostomy tube prevents excessive penetration of the tube into the trachea. However, when using a tracheostomy tube having a removable flange as described hereinabove, the flange will not normally have been attached to the tube at the time that the tube is introduced into the body (to permit easy removal of the introducer sheath following insertion of the tracheostomy tube). Therefore, without the presence of the flange to prevent undue penetration, the tracheostomy tube might inadvertently be inserted an excessive distance into the trachea. If this occurs, there is often no convenient way to retrieve the tube without causing additional trauma to the patient.

Therefore, a lock mechanism is provided to prevent such excess penetration of the tracheostomy tube. In the embodiment shown in FIG. 7, the lock mechanism comprises a stop mechanism 72 that is provided on dilator 70. A securement member 74 is provided for engagement with the stop mechanism. In this embodiment shown, the stop mechanism is an annular ring and the securement member is a rotatable cap. Stop mechanism 72 is connected to dilator 70 by a friction fit or other attachment mechanism that substantially limits or prevents altogether axial movement of the stop mechanism along the dilator when an axial force is applied to the stop mechanism. Alternatively, the stop mechanism can be molded as an integral part of the dilator.

Preferably, the securement member includes screw threads 76 or like attachment mechanisms for locking the securement member to a complementary attachment site on the tracheostomy tube, such as collar 22. FIG. 8 shows a side view of the attachment of the securement member to collar 22, and FIG. 9 shows a sectional view of this connection. Those skilled in the art will recognize that other locking elements could be readily substituted for the stop mechanism and securement rings, as long as these elements cooperate in a manner to substantially limit or prevent altogether axial movement of the stop mechanism along the dilator, thereby preventing excess penetration of the tracheostomy tube.

If the tracheostomy tube is inadvertently inserted too far when the dilator and tracheostomy tube are connected in this manner, the physician can retrieve the tracheostomy tube from the body by simply pulling back on the loading dilator 70. Once tracheostomy tube 20 is in proper position in the body, the securement cap can be disconnected from the tracheostomy tube. The loading dilator and the securement cap are then removed in tandem, and the attachable flange can be connected to the tracheostomy tube.

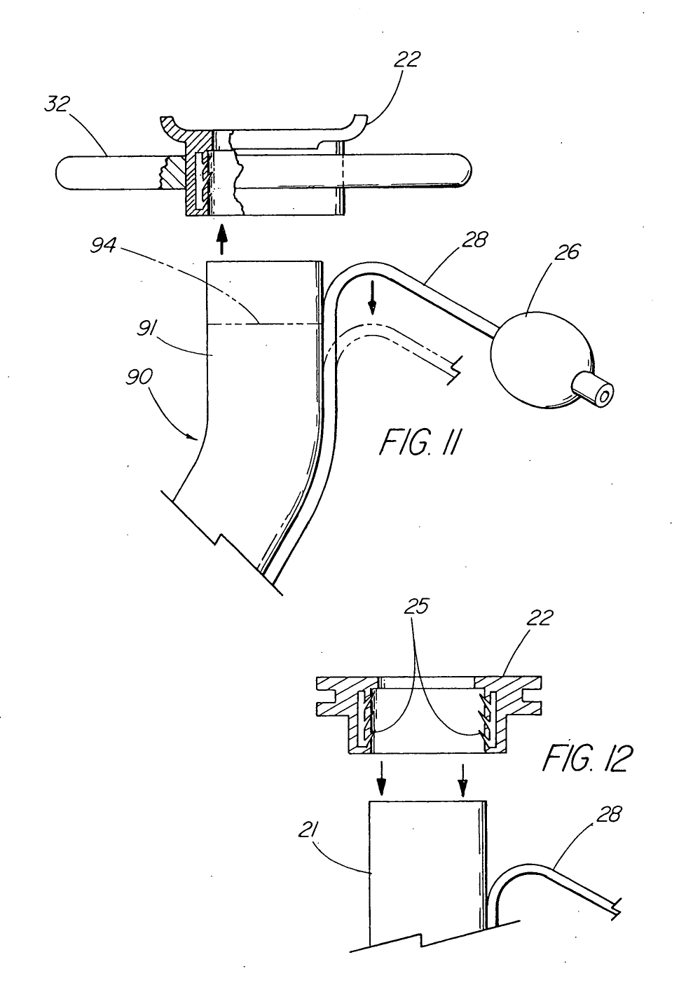

The tracheostomy tube may also be provided with an optional inner cannula. Since secretions such as mucus and phlegm can partially or completely obstruct a tracheostomy tube, it is known to provide some tracheostomy tubes with a removable inner cannula. The inner cannula 80 is inserted into the main lumen of the tracheostomy tube 20, and may be separately removed from the tracheostomy tube for cleaning of these obstructions or for replacement. As a result, the main body (outer cannula) of the tracheostomy tube that has flange 32 attached thereto can generally be left in the trachea for longer periods of time than might otherwise be possible. An arrangement comprising a removable inner cannula 80, tracheostomy tube 20, and removable flange 32 is illustrated in FIG. 10.

Another feature of the present invention comprises a trimmable tracheostomy tube 90. In critical care applications, which by their nature must generally be performed as rapidly as possible, tracheostomy tubes of optimal size for each and every patient may not always be readily available. As a result, a clinician must sometimes utilize the best available tracheostomy tube, even if it isn't optimal for a particular patient. The trimmable tube of the present application is variable in length, so that it can be used in a wider variety of patients than existing tubes. One embodiment of a trimmable tube is shown in FIG. 11. This tube has a length greater than that which might be suitable for a particular use.

After inserting the tracheostomy tube, a clinician can trim the tube along a trim line, preferably at the proximal end of the tube, to shorten the tube to a desired length to more optimally fit the patient. With this concept, inflation line 28 of the tracheostomy tube 90 can be peeled-away from main body 91 of tube 90 to allow the tube to be trimmed to a desired length. As used herein, the term “trim line” refers to any surface of the tracheostomy tube that is cut, or trimmed, through by the clinician in order to shorten the length of the tube to fit a particular patient. An example of a trim line 94 is shown by the broken line in FIG. 11. Tube body 91 may be trimmed along trim line 94 by conventional means such as a knife, scissors, etc., to reduce the length of tube body 91 to an appropriate size for the patient. For convenience, marked trim lines may be indicated on the outer surface of tube body 91. However, there is no requirement that trim lines be separately marked, and the tube may be trimmed to any length along trim lines identified and defined by the clinician at any point along the length of the tube. After the tube is trimmed as described, flange 32 may be engaged with the tracheostomy tube by any convenient means, preferably in combination with collar 22, as shown in FIG. 11.

The tracheostomy tube, dilator and their various parts are made from well-known materials that are commonly employed for such use.

While this invention has been particularly shown and described with reference to preferred embodiments thereof, it will be understood by those skilled in the art that various changes in form and details may be made therein without departing from the spirit and scope of the invention. Those skilled in the art may recognize or be able to ascertain using no more than routine experimentation, many equivalents to the specific embodiments of the invention described specifically herein, which equivalents are intended to be encompassed in the scope of the invention.

What is claimed is:

1. A tracheostomy tube comprising:
a hollow tubular body having a proximal end portion, a distal end portion and a curved portion intermediate said proximal and distal end portions; and
a flange situated at said proximal end portion, said flange being capable of radial extension from said tube, and being manipulatable to selectively prevent said radial extension.

2. The tracheostomy tube of claim 1, wherein said flange is selectively attachable to said proximal end portion to provide said radial extension and detachable from said proximal end portion when said radial extension is to be prevented.

3. The tracheostomy tube of claim 2, wherein said flange is attachable to said tube by a snap-fit.

4. The tracheostomy tube of claim 3, wherein said hollow tubular body includes a collar at its proximal end, said collar having a groove, and wherein said flange includes a cut-away portion, said cut-away portion and said groove being cooperatively sized and shaped to mate when said flange is attached to said tube.

5. The tracheostomy tube of claim 4, wherein at least one snap is provided at said cut-away portion to provide said snap-fit with a receptacle on said tubular body.

6. The tracheostomy tube of claim 4, wherein said collar is integral with the hollow tubular body.

7. The tracheostomy tube of claim 4, wherein said collar includes one or more barbs for attaching the collar to said hollow tubular body.

8. The tracheostomy tube of claim 1, further comprising a removable inner cannula insertable in said hollow tubular body.

9. The tracheostomy tube of claim 1, wherein said hollow tubular body includes an inflatable cuff surrounding a part of said distal end portion, said tracheostomy tube further comprising an inflation line connecting said cuff to a source of an inflation fluid.

10. The tracheostomy tube of claim 9, wherein said inflation line is connected to said hollow tubular body along a length of said body, at least a portion of said inflation line being selectively peelable from said hollow tubular body.

11. The tracheostomy tube of claim 10, wherein said hollow tubular body is trimmable along a trim line to reduce the length of said body, and said inflation line is peelable at least to a point below said trim line.

12. An insertion device comprising a tracheostomy tube and a loading dilator, the tracheostomy tube having a longitudinal bore and a tapered distal tip; the loading dilator having a larger-diameter stepped proximal portion and a smaller diameter distal portion tapered at its distal end, the smaller-diameter distal portion being sized to be insertable through the longitudinal bore of said tracheostomy tube such that said tapered distal portion extends axially beyond the tapered distal tip of the tracheostomy tube, the tracheostomy tube having a proximal end and further comprising a stop portion at said proximal end for engaging a distal portion of the larger-diameter stepped portion of the dilator to limit axial movement of the loading dilator through the tracheostomy tube.

13. The insertion device of claim 12, wherein said stop portion comprises a collar provided at said proximal end of said tracheostomy tube.

14. The insertion device of claim 12, wherein said stop portion comprises a larger-diameter proximal end portion of the tracheostomy tube.

15. The insertion device of claim 12, wherein said tapered distal portion of said dilator is complementary to the tapered distal tip of the tracheostomy tube such that a generally smooth conical insertion tip is defined thereby.

16. The insertion device of claim 15, wherein said generally smooth conical insertion tip has a profile sufficient for dilating an opening in the body of a patient for insertion of said tracheostomy tube.

17. The insertion device of claim 12, wherein said larger-diameter stepped proximal portion of the dilator comprises a gripping surface.

18. The insertion device of claim 17, wherein said gripping surface is formed from one or more polymers.

19. The insertion device of claim 17, wherein said gripping surface is formed from a member selected from the group consisting of lower durometer urethanes, thermoplastic rubbers, thermoplastic elastomers and non-thermoplastic elastomers.

20. The insertion device of claim 12, wherein said dilator larger-diameter stepped proximal portion and smaller diameter distal portion comprise integral molded components.

21. The insertion device of claim 12, wherein said larger-diameter stepped proximal portion of said dilator includes a longitudinal passageway, and a portion of said smaller diameter distal portion is securely received within said longitudinal passageway.

22. A device for percutaneous insertion into the trachea of a patient, comprising:
a tracheostomy tube having a longitudinal passageway therethrough, said tracheostomy tube having a distal end portion percutaneously insertable into said trachea and a proximal end portion exterior to the trachea when said distal end portion is inserted; said tracheostomy tube further having a radially extending flange attachable to said proximal end portion of the tracheostomy tube after said distal end portion has been inserted into the trachea;
a dilator positionable within said longitudinal passageway of said tracheostomy tube for dilating an opening in said trachea for insertion of said tracheostomy tube; and
a locking assembly for locking the tracheostomy tube to the dilator during insertion of said tracheostomy tube into the trachea.

23. The device of claim 22, wherein said locking assembly comprises a securement member associated with the dilator, said securement member engageable with a complementary member on said tracheostomy tube.

24. The device of claim 23, wherein said locking assembly further comprises a stop member disposed on an outer surface of said dilator, said stop member engaged with said dilator such that substantial axial movement of said stop member along said dilator is prevented when an axial force is applied to said stop member, said stop member positioned on said outer surface and engageable with said securement member and said complementary member for preventing excess penetration of the tracheostomy tube into the trachea.

25. The device of claim 24, wherein said stop member comprises an annular ring integral with said dilator.

26. The device of claim 24, wherein said stop member comprises an annular ring fitted on the outer surface of said dilator.

27. The device of claim 24, wherein said complementary member comprises a collar integral with said tracheostomy tube.

28. The device of claim 24, wherein said complementary member comprises a collar fitted on an exterior surface of said tracheostomy tube.

29. The device of claim 24, wherein said securement member comprises a cap member having one or more screw threads engageable with said complementary member for locking said tracheostomy tube to said dilator.

30. A method of inserting a tracheostomy tube into the trachea of a patient, the method comprising:
providing a tracheostomy tube comprising a hollow tubular body having a longitudinal passageway therethrough, said tubular body having a distal end portion for insertion into the trachea, and a proximal end portion exterior to the trachea when the distal end portion is inserted, said tubular body further having a curved portion intermediate said proximal and distal end portions;
inserting said distal end portion of said tubular body into said trachea;
trimming an excess portion of said proximal end portion of said tubular body; and
engaging a flange with said tracheostomy tube at said proximal end portion of said tubular body.

31. The method of claim 30, wherein said flange is attached to a collar, said collar being connected to said proximal end portion.
